(19) United States
(12) Patent Application Publication
(10) Pub. No.: US 2015/0073526 A1
(43) Pub. Date: Mar. 12, 2015

(54) Retractable Flow Maintaining Stent

(71) Applicant: Bryan W Kluck, Allentown, PA (US)

(72) Inventor: Bryan W Kluck, Allentown, PA (US)

(21) Appl. No.: 14/020,855

(22) Filed: Sep. 8, 2013

(51) Int. Cl. A61F 2/90; A61F 2/966

(52) U.S. Cl. CPC A61F 2/90; A61F 2/966
USPC 623/1.12

(57) ABSTRACT

A self-expanding stent is delivered to an affected site within the human body with a guide wire and hand-manipulable control apparatus. The stent is capable of full expansion along a pre-determined length of its body with the remainder of the overall length conically tapering to a fixed connection point with the guide wire with a flexible joint between the sections. A covering sheath delivers the stent to the affected area with a tip capable of penetrating a blockage or obstruction in a vessel such that the stent can be exposed to begin its expansion. Once the obstruction is opened, the stent can be recaptured or retrieved by pulling it back into the sheath, collapsing the expanded stent with the assistance of the conical portion and flexible joint, and withdrawing the stent and wire completely from the vessel. A rotating motion will cause the conical and cylindrical sections of the stent to separate at the flexible joint.

### CROSS-REFERENCE TO RELATED APPLICATION

The instant application claims priority to, and incorporates expressly by reference, U.S. Non-Provisional patent application Ser. No. 12/395,694, filed on Oct. 26, 2010, as if fully set forth herein, which application was published as U.S. Patent Application Publication No. US 2012/0101560 A1 on Apr. 26, 2012, and which remains pending as of the date of filing of this application.

### BACKGROUND OF THE INVENTION

This invention relates to the deployment and retrieval of a flexible, self-expandable and retractable multi-part stent for use in a body passageway, including an elongated tubular wall catheter with a guide wire and hand manipulable control apparatus.

Use of expandable stents is known for treatment and repair of damaged areas of the circulatory system of the body such as blood vessels. They can be implanted in a human patient's blood vessels to maintain the free, unobstructed passage of bodily fluids. Stents have been used to reinforce weakened body lumens such as the urethra, bile ducts, blood vessels, the trachea, coronary arteries, and the esophagus. Stents are generally cylindrically shaped devices and are typically implanted within a vessel in a contracted state and expanded when in place in the vessel in order to maintain an unobstructed passage within the vessel to allow fluid flow through the vessel. As the stent is positioned it self-expands to conform to the inside contour of the vessel wall. The delivery system is then withdrawn and the expanded stent holds the blocked vessel open.

Another commonly available device is the balloon stent. Balloon-expandable stents (BES) are mounted in their reduced diameter state on nylon or polyethylene balloons, usually by manual crimping, while others are available pre-mounted. The balloon catheter is inserted through the blood vessels, across the blockage, and is inflated to open the blockage. A pre-loaded delivery system is inserted through the same path as the balloon catheter and carries the stent to the blockage site; the stent is then deployed across the blockage. Stents have been implanted by mounting the stent on a balloon portion of a catheter, positioning the stent in a body lumen at the stenosis, and expanding the stent to an expanded state by inflation of the balloon within the stent. The stent remains in place as the balloon is deflated and removed with the catheter. Some may also be removed by use of grasping tools.

Presently available stents include self-expanding stents, which will automatically deploy as their constraining sheath is pulled back beyond a certain point. Once deployed beyond the point where automatic expansion occurs, theses stents usually cannot be recaptured without risk of injury, if ever. The deployed stent serves as a foreign object within the circulatory system that is recognized as such and attacked by platelets. This in turn begins the clotting process which may, again, occlude the vessel. In order to retard this unwanted consequence, anti-platelet agents must be given for a period of months to years. This is not optimal, particularly in the brain where the chance of bleeding is very high.

The balloon stents at present are of limited use in most delicate procedures because the high pressure inflations necessary to achieve full stent exposure are not possible in the intracranial vascular system due to the size of the balloon stent and the small delicate arterial structure. While they may restore the lumen, the balloons that have the diameter and flexibility to get into the cerebral circulations do not allow for fluid flow when expanded. The inflated balloon obstructs the flow. Therefore, a need exists for a structure much smaller than a balloon stent for use in intracranial procedures.

The problems with the current commercially available stents include: the potential for crushing from external pressure, risk of tearing the interior of vessel walls or penetrating a vessel wall upon retraction, and the inability to allow passage of the stent beyond an obstruction. Additionally, drug eluting balloons mostly obstruct flow which limits their time in contact with the vessel wall and thus the effectiveness of the drugs.

Some examples of currently available stent-like apparatus are described with reference to the following publications. U.S. Pat. No. 6,533,810 [Hankh, et al.], U.S. Patent Application Publication US 2001/0010013 A1 [Cox, et al.], and U.S. Patent Application Publication US 2006/0184226 A1 [Austin] all disclose self-expandable stents with a tapered or cylindrical geometry. None of these references, however, describe a means for relocating, retrieving or removing the stent once it is in position within a blood vessel or otherwise. The present invention is capable of being removed once its use is complete by retracting the micro-stent into the delivery vehicle, such as a micro catheter, without any damage to the vessel walls.

U.S. Pat. No. 6,676,692 [Rabkin, et al.] and U.S. Pat. No. 7,258,696 [Rabkin, et al.] both disclose an apparatus for delivering and retrieving a stent which can be accomplished by a balloon attached to a catheter, a self expanding type that radially expands once deployed from the distal end portion of a delivery catheter, to a desired location in body vessels by which the stent can be positioned. The stents disclosed in the publications are collapsible for ease of removal. Rabkin employs capturing wires which extend parallel to the frame wires but outside the balloon to help retrieve the self-expanding stent. The potential for injury suggests that the wires of Rabkin may catch on the inner walls of the body vessel causing tears and bleeding during the retrieval process. Moreover, these stents may be useful only for blood vessels and other tubular or elongated cylindrical body structures such as the esophagus, bile ducts, urinary tract, intestines or trachea-bronchial tree where there may be room for a balloon type stent. None of the above mentioned references may be useful for intracranial use like the present invention because of the high pressure involved with balloon stents; it would be too dangerous to inflate and, once inflated, would block flow of the blood because of the small size of the intracranial vessels.

U.S. Pat. No. 6,821,291 [Bolea, et al.] and U.S. Pat. No. 6,569,181 [Burns] disclose methods for retrieval of a collapsible stent, through the usage of maneuverable or manipulable tool which aids the physician operating to compress the stent from its expanded diameter and retrieve the stent from a vessel. Both patent references use a tubular stent made up of a mesh pattern which can be metallic or non-metallic. Bolea achieves removal by use of a tool having a grasping attachment which may catch on the inner walls of the body vessel causing unwanted tears and bleeding during the retrieval process. Neither of the references uses a conical or tapered cylindrically shaped stent as does the present invention to ease in the retrieval or repositioning of the stent. Nor do they disclose the capability of the stent to carry and be used in conjunction with pharmacological agents.

U.S. Pat. No. 5,961,547 [Razavi], U.S. Pat. No. 5,441,516 [Wang, et al.], U.S. Pat. No. 5,449,372 [Schwaltz, et al.] and U.S. Pat. No. 5,411,549 [Peters] disclose temporary or retractable stents in the shape of a spiral coil or double helix. Although these stents are made of different materials, such as metal or plastic, and have differences in the techniques of their deployment (heat-activated, self-expanding, or balloon expandable), as well as methods for their retrieval (mechanical straightening vs. softening by increasing temperature vs. latch retraction), all of them have one common feature. The stents are connected with a wire extending outside the patient at all times and when they have to be removed, they are simply retracted back into the catheter with or without prior preparation for retraction and removal. For this reason these stents cannot be left in the human body for more than a very short time. The connecting wire can traverse the entire body increasing risk of thrombus formation around the wire and distal embolization, and the onset of infection.

U.S. Pat. No. 6,007,573 [Wallace, et al.] for “Intracranial Stent and Method of Use” discloses a stent catheter for intra-cranial use. The stent is a rolled sheet stent and releasably mounted on the distal tip of the catheter with a low profile retaining tab. Wallace does not employ the use of a sheath that stays around stent. The Wallace stent can be temporarily placed, or permanently placed. However, to rewind, reposition or remove the Wallace device, grasping tools must be used. The present invention allows the stent to be retracted and removed easily without the use of grasping tools. Further, it may be advanced without a micro catheter and the stent will span the obstruction pushing the obstructing material aside and allowing flow to be restored. For retraction and removal the cover or sheath may be easily re-advanced to re-capture the stent wire for easy removal.

U.S. Patent Application Publication No. US 2009/0105737 A1 [Fulkerson, et al.] for “Acute Stroke Revascularization/Recanalization Systems Processes and Products Thereby” discloses a deployable stent for use in revascularization of a human blood vessel. The stent is composed of nitinol or spring tempered stainless steel expandable mesh and is self-expanding device once released from a transport catheter. Fulkerson claims that the stent device is reconstrictable and/or retrievable but fails to describe the procedure for doing so or any of the elements of the device that facilitate the process of recapture of the stent device from deployment. The only insight in the precise structure of the stent device is the expandable mesh material and that the mesh must be in a pattern that enables the device to be fully retracted into the catheter. Although not specifically stated, it appears that retraction is accomplished by pulling the stent device back into the transport catheter. There are no specific parts or segments of the stent device described that permit either maximum length deployment or assistance in retrieval and removal from the vessel subsequent to the revascularization of the vessel without injury to the vessel walls.

None of the known stents discussed above permit complete wall apposition at the distal and proximal ends, even when fully expanded. These stents have device geometries with restricted expansion properties along their length limited by the proximal end being permitted to expand only into a conical, rather than substantially complete cylindrical, shape from the fixed connection to the axially positioned control wire, yet still permit the flow of bodily fluids through the partially expanded stent form, and have the capacity to be collapsed and be withdrawn without the need for recapture tools. Also, none of the known stents permit the full and complete expansion of their distal end portions to maximally revascularize the vessel.

Accordingly, it is an object of the present invention to provide a new and improved flow maintaining stent wire which can be easily delivered and retracted while still maintaining blood flow in body vessels during a medical procedure. Another object of the present invention is to provide a new and improved flow maintaining stent wire which has a construction to allow the passage of the device beyond an obstruction in the human circulatory vessels.

Still another object of the present invention is to provide a new and improved flow maintaining stent wire which is capable of treating an obstruction with either direct pressure or pharmacological coating. Yet another object of the present invention is to provide a new and improved flow maintaining stent wire which allows the delivery and direct administration of any desired and suitable pharmacological agent.

Still another object of the present invention is to provide a new and improved flow maintaining stent wire having different geometries at its distal and proximal portions for enhancing the enablement of retrieval or recapture, and removal of the stent wire without leaving any foreign substances that may stimulate internal hyperplasia, sub-acute or acute thrombosis, etc.

Other objects will appear hereinafter.

### SUMMARY OF THE INVENTION

The present invention is a self-expanding stent delivered to the affected site within the human body with a guide wire and hand-manipulable control apparatus. The apparatus may be further described as a self-expanding stent with the particular geometry and cross-wire configuration dependent upon the area of use within the human body, that is capable of full expansion only along a pre-determined length of its body with the remainder of the overall length tapering (in substantially conical form) to a fixed connection point with the guide wire. The stent is delivered to the affected area in a covering sheath that has a tip that is capable of penetrating a blockage or obstruction in a vessel such that the stent can be exposed to begin its expansion. Once expanded across the vessel, the interstitial spaces between the stent wire-form permit bodily fluids to begin to flow again beyond the point of obstruction.

Once the obstruction has been opened and flow reestablished, the stent can be recaptured or retrieved by pulling it back into the sheath and withdrawing the stent wire completely from the vessel. The outer surfaces of the stent can be coated with suitable pharmaceutical agents to locally treat the area of the occlusion or obstruction, rather than give much larger doses of the same agent through the circulatory system, thus greatly reducing the risk of bleeding in the affected area, as well as other unintended locations in the body.

Further, the present invention could elute a lytic agent such as tissue plasminogen activator (tPA) or Urokinase directly on the obstructing thrombus. This could significantly reduce the systemic tPA release and also the downstream tPA that would flow to the injured tissue. The stent portions of the present stent wire invention may be coated with a variety of therapeutic agents to aid in the treatment of the damaged tissue.

The present invention is directed to a retractable self-expanding stent apparatus for re-establishing fluid flow in a body vessel while maintaining fluid flow within the vessel during use including a self-expanding stent with proximal and distal ends and being partitioned into a full expansion portion and a partial expansion portion. The full expansion portion is configured as a cylindrical section and located at the distal end of the stent and the partial expansion portion is configured as a conical section and located at the proximal end of the stent. The cylindrical and the conical sections are joined at their juxtaposed ends by a common flexible reinforcing joint, which joint permits the independent staged expansion of the cylindrical portion to its full diametric expansion prior to and continuing as the conical section reaches full expansion and which joint permits the collapse of the conical section prior to and continuing through the staged collapse of the cylindrical section. The common joint has the same minimum and maximum diametric dimension for both the cylindrical section and the conical section of the stent. A guide wire is attached to the proximal end of the stent for controlling the positioning of the stent and at least one covering sheath is used for retaining and transporting the self-expanding stent and guide wire to the affected area for treatment.

The stent has a predetermined geometry of cross-wire mesh-like configuration having sufficient interstitial spacing to permit the re-establishment of bodily fluid flow depending upon the area of use within the human body. The stent is capable of self-expansion upon deployment and self-collapse upon retraction as the stent is respectively pushed out of and pulled into the covering sheath. The stent is also capable of self-collapse upon retraction as the covering sheath is pushed over the stent and self-expansion upon deployment as the stent is pushed out of the covering sheath.

The stent device may also include a tip located at the distal end of the stent wire which is capable of penetrating a blockage or obstruction in a vessel. The stent is capable of expanding entirely across a vessel at deployment causing the material of the blockage to be pushed against the vessel walls and to immediately permit the re-establishment of bodily fluid flow again beyond the point of obstruction. The stent is also capable of being coated with one or more pharmaceutical agents to locally treat the obstruction in a vessel. In the event that the stent becomes trapped or engaged with the vessel wall the conical and cylindrical sections can be separated. The common flexible reinforcing joint has a lesser tensile strength than the conical or cylindrical sections of the stent such that a rotating or twisting motion will cause the conical and cylindrical sections to separate at the flexible joint. Thus, the cylindrical section can remain in place in the vessel without impeding fluid flow and the conical section can be withdrawn.

Also described is a method for the deployment and recapture of a retractable self-expanding stent apparatus for re-establishing fluid flow in a body vessel while maintaining fluid flow within the vessel during use which includes a self-expanding stent that has a proximal and distal end and being partitioned into a full expansion portion and a partial expansion portion, the full expansion portion configured as a cylindrical section and located at the distal end of the stent and the partial expansion portion configured as a conical section and located at the proximal end of the stent. The cylindrical and conical sections being joined at their juxtaposed ends by a common flexible reinforcing joint, which joint permits the independent staged expansion of the cylindrical portion to its full diametric expansion prior to and continuing as the conical section reaches full expansion and which joint permits the collapse of the conical section prior to and continuing through the staged collapse of the cylindrical section, such that the common joint has the same maximum diametric dimension for both the cylindrical section and the conical section of the stent. Also included is a guide wire attached to the proximal end of the conical section of the stent with all placed inside a catheter or sheath. The sheath with said stent and guide wire attached are positioned at the point of obstruction in a vessel containing bodily fluids of a human and the guide wire is advanced outward from the distal end of the sheath exposing the cylindrical section of the stent beyond the common joint so that said stent will begin its self-expansion as the conical section is further exposed and is permitted to expand beyond the diametric confining dimension of said sheath. Once the treatment has been accomplished the guide wire is withdrawn inward away from the distal end of the sheath, contacting the conical section of the stent against the distal opening of the sheath and forcing the reduction in the diametric measurement of the common joint by the sliding contact of the conical section of the stent against the inner circumference of the sheath resulting in the collapse of the stent to a size capable of being completely drawn into the sheath.

An alternate method for deployment using the same elements may be achieved by pulling the sheath back and away from the stent along the guide wire, exposing the cylindrical section of the stent beyond the common joint so that the stent will begin its self-expansion as the conical section is further exposed and is permitted to expand beyond the diametric confining dimension of said sheath. Retraction can be accomplished by pushing the sheath along the guide wire, contacting the conical section of the stent against the distal opening of the sheath and forcing the reduction in the diametric measurement of the common joint by the sliding contact of the conical section of the stent against the inner circumference of the sheath resulting in the collapse of the stent to a size capable of being completely drawn into the sheath.

Both deployment/retraction methods may be augmented by placing the stent and connected guide wire positioned inside the sheath within another catheter or sheath. The stent may also be coated with one or more pharmaceutical agents to locally treat the obstruction in a vessel. An extended tip may be placed at the distal end of the stent wire that is capable of penetrating a blockage or obstruction in a vessel. In the event that the stent becomes trapped in or engaged with a vessel wall separation of the conical and cylindrical sections of the stent can be achieved by a rotating or twisting motion such that the separation will occur at the common flexible joint allowing the conical section to be withdrawn and the cylindrical section to remain in place in the vessel.

The present invention is a stent wire device and manually controlled capture sheath of a sufficient size to allow for the constant access of a surgeon to the affected area, thus allowing further work to treat the affected area including but not limited to balloon, stent or other mechanical device delivery.

### BRIEF DESCRIPTION OF THE DRAWINGS

For the purpose of illustrating the invention, there is shown in the drawings forms which are presently preferred; it being understood, however, that the invention is not limited to the precise arrangements and instrumentalities shown.

FIG. 1 is an isometric view of a catheter containing the stent wire of the present invention.

FIG. 2 is an enlarged cross-sectional view of the undeployed retractable flow maintaining stent wire of the present invention showing both proximal and distal sections thereof, with a flex-joint disposed between each of the sections.

FIG. 3 is an enlarged sectional view of the retractable flow maintaining stent wire of the present invention deployed within a partially clogged circulatory vessel showing the maximal deployment and outward compression of the distal section, with the proximal section retaining its conical shape for assisting in retrieval.

FIG. 4 is an enlarged sectional view of the retractable flow maintaining stent wire of the present invention positioned within a constraining sheath within the catheter of FIG. 1 awaiting deployment showing the flex-joint separating the proximal and distal sections.

FIG. 5A is a side view of an elongated sheath containing a constrained stent wire of the present invention.

FIG. 5B is a side view of the stent wire of the present invention in a deployed state with the constraining sheath retracted along almost the entire length of the stent wire.

FIG. 5C is a side view of the constraining sheath and stent wire of FIG. 5C with the obdurator removed.

FIG. 5D is a side view of the initiation of the recapture of the stent wire of the present invention re-entering the constraining sheath.

FIG. 5E is a side view of the stent wire of the present invention recaptured and collapsed within the constraining sheath.

### DETAILED DESCRIPTION OF THE PREFERRED EMBODIMENTS

The following detailed description is of the best presently contemplated mode of carrying out the invention. The description is not intended in a limiting sense, and is made solely for the purpose of illustrating the general principles of the invention. The various features and advantages of the present invention may be more readily understood with reference to the following detailed description taken in conjunction with the accompanying drawings.

Various embodiments of the retractable flow maintaining stent wire for delivering and retracting a self-expanding stent in accordance with the invention are described herein. Referring now to the drawings in detail, where like numerals refer to like parts or elements, there is shown in FIG. 1 a catheter 10 including a manifold 30 with a main port 32 and auxiliary port 32a. Mounted through the main port 32 is a guide wire 12 that exits through the manifold 30 and through the hollow shaft 16. At the distal end of the shaft 16 the guide wire 12 can exit the shaft 16. Mounted to the distal end of the stent wire 12 is a self-expanding stent 14 of the present invention that can be delivered, using the apparatus described, to the affected site within the human body. The stent 14 is manipulable into and out of the shaft 16 with the use of the guide wire 12 and hand-manipulable control apparatus 28 located above the main port 32.

The stent or micro-stent 14 has the particular geometry and cross-wire configuration dependent upon the area of use within the human body, that is capable of full expansion only along a pre-determined length of its body with the remainder of the overall length tapering in substantially conical form to a fixed connection point 22 to connect with the guide wire 12. The stent 14 is fully releasable if necessary during a procedure in order to prevent an arterial tear as described in greater detail below.

The micro-stent 14 has a proximal end 18 and a distal end portion 20. The proximal end 18 of the stent 14 is configured as an expanding conical section 24 emanating forward from the connection point 22 with the guide wire 12 to the fully expanding section 26 that extends along the remainder of the stent 14 to its distal end 20. Between the two sections 24 and 26 is a flex-joint 25 that joins the sections 24, 26 together in a longitudinal arrangement and permits each section to completely form its respective geometric shape.

The guide wire 12 is manipulable within the shaft 16 and connects the stent 14 upward through the hollow shaft 16 to the control apparatus 28. The catheter 10 is constructed and arranged to allow longitudinal movement of the guide wire 12 along and through the shaft 16 to effect the longitudinal movement of the stent 14 through the shaft 16 and to permit the deployment by movement outward from the end of the shaft 16 and retraction by movement inward into the end of the shaft 16. The longitudinal movement of the guide wire 12 within the shaft 16 in the deployment or outward direction allows the stent 14 to exit the restraining sheath or shaft 16 and permits the stent outwardly expanding or compression section 26 to expand from its contracted, restrained state as it exits the shaft 16. As the distal section 26 fully expands to form its expanded substantially cylindrical shape for compressing against the vessel walls, the proximal section 24 begins its expansion into a substantially conical shape matching the fully deployed cylindrical diameter of the distal section 26 and tapering down to a point connection to the stent wire connection point 22 at its extreme proximal end.

An opposing longitudinal movement of the guide wire 12 within the shaft or sheath 16 in the withdrawal or inward direction forces the proximal section 24 of the stent 14 to initiate collapse of the distal section 26 of the stent 14. In performing the inward motion of the guide wire 12 the conical section 24 begins to collapse the cone at the proximal end of the stent 14 and, as connected to the distal section through the flex-joint 25, causes the distal section 26 to independently contract to its constrained configuration allowing both section 24, 26 to reenter the sheath 16. The manifold 30 also provides for a secondary port 32a in which pressure monitoring, fluid medicaments may be administered, or be used in aspirating away particles removed by the procedure.

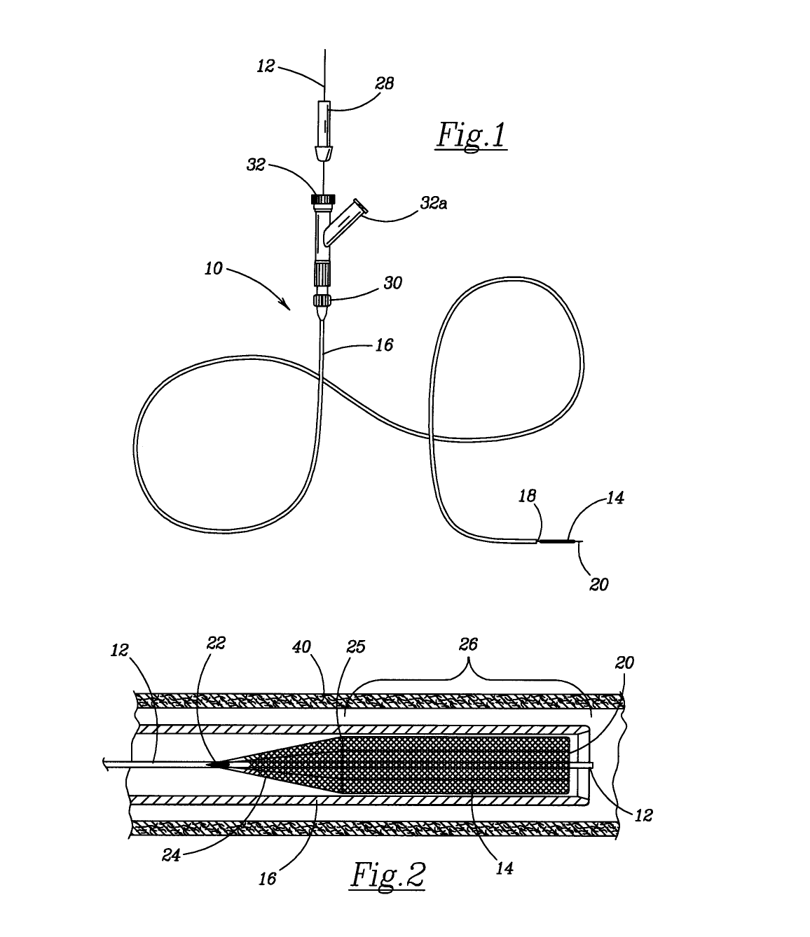

FIG. 2 shows an enlarged cross-sectional view of the un-deployed retractable flow maintaining micro-stent 14 of the present invention that has been delivered to the affected area in a covering sheath or catheter tube 16 within a vessel wall 40 of an artery, vein or other like vessel within the human body. The sheath 16 maintains the constrained micro-stent 14 in its retracted, collapsed state at or near its distal end. The guide wire 12, to which the micro-stent 14 is attached, extends a short distance beyond the distal end of the stent 14 as a lead to penetrate an obstruction in the vessel in which the stent 14 is to be deployed. In the restrained or collapsed state, the stent 14 extends along the guide wire 12 a short distance beyond the distal end of the stent 14 as the stent 14 collapses forward from the proximal end 18 at the connection point 22. Upon deployment from the sheath 16, the micro-stent 14 is capable of penetrating a blockage or obstruction in a vessel such that the stent 14 may be pushed through the obstruction as it begins its expansion. The micro-stent 14 may be composed of a metallic or polymeric mesh material to allow the flow of bodily fluids through the expanded stent 14 without substantial blockage or interference in the fluid flow. These materials are intended to have sufficient material memory to permit both the full expansion and contraction in their respective complete deployed and retracted positions.

The micro-stent 14 is constructed in two sections; a cylindrical, fully expandable section 26 and a conical partially expandable section 24 joined together by a flexible joint 25 that permits independent radial motion of each section. Both sections 24, 26 are constructed of an interlaced wire mesh that is reinforced along the longitudinal direction for strength. The circumferential flexible joint 25 is positioned between the two sections 24, 26 to provide for and not to interfere with the independent expansion or contraction of either section. However, as with any joint, the presence of the flexible joint 25 will cause one section to follow any extreme outward or inward radial motion of the other section. In this way the flexible joint 25 allows for the almost complete expansion of the distal section 26 while the proximal section 24 is still being pushed out of the sheath 16 such that as the proximal section 24 exits the end of the sheath 16 that section expands independently of the already expanded distal section 26. In the withdrawal mode, the proximal conical section 24 is drawn into the sheath 16 collapsing the cone-like shape quickly which, in turn, draws the flexible joint 25 away from the vessel wall 40. As the proximal section 24 is drawn into the sheath 16 the portion of the distal section 26 closest to the flexible joint 25 is also reduced in radial dimension drawing that portion away from the vessel wall 40 and initiating the collapse of the expanded cylindrical shape along the remainder of the distal section 26 in stages as the stent 14 is drawn back into the sheath 16.

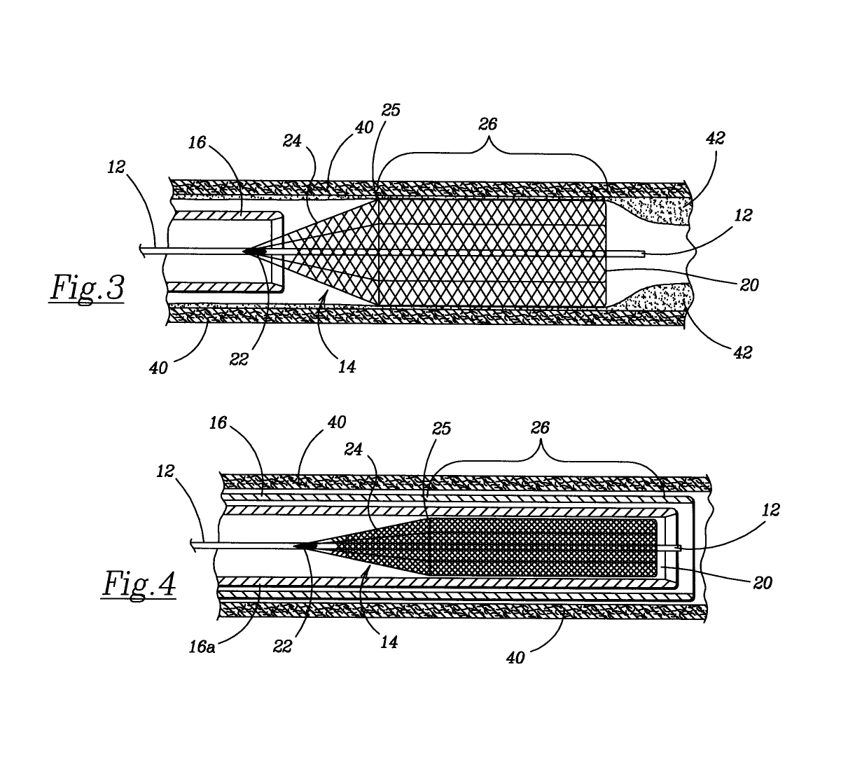

Referring now to FIG. 3 there is shown an enlarged sectional view of the retractable flow maintaining stent 14 of the present invention deployed within a partially clogged circulatory vessel 40. Once expanded across vessel 40, as shown in FIG. 3, the interstitial spaces between the interlaced mesh of stent 14 permit bodily fluids to flow again beyond the point of obstruction. The stent is shown partially opening a partly occluded vessel 40 with built up plaque 42 shown beyond the distal end 20 of the expended stent 14 and against the walls of vessel 40. Once the obstruction has been sufficiently opened, the stent 14 can be recaptured or retrieved by using the guide wire 12 to pull the stent 14 back into the sheath 16 causing the collapse of the expanded sections 24, 26 as described above. The first to contact the sheath 16 upon retracting is the conical section 24 that is caused to collapse as it is pulled into the sheath 16. As the conical section 24 is reduced in its overall diameter, with such diametric reduction extending to the flexible joint 25 and along the cylindrical section 26, the cylindrical section 26 follows the contraction of the flexible joint 25 and the conical section 24 contracting to its original restrained dimensions for fitting within the sheath 16. The collapse of the expanded stent 14 is complete when both sections 24, 26 have been drawn back into the sheath 16. Once the withdrawal is completed, the stent 14 may either be repositioned for continuing to work to open the obstruction by repositioning, or be withdrawn entirely from the vessel.

It has been shown to be effective for the outer surfaces of the stent 14 to be coated with one or more suitable pharmaceutical agents to locally treat the tissues of obstructed or occluded area, rather than give much larger doses of the same agent by other means. The stent 14 of the present invention could be coated with a lytic agent such as tissue plasminogen activator (tPA) or Urokinase for directly coming into contact with the obstructing thrombus. This could significantly reduce the systemic tPA release and also the downstream tPA that would flow to the injured tissue. This will greatly reduce the risk of unrestrained bleeding in the affected area, as well as other unintended locations in the body.

Referring to FIG. 4, an enlarged sectional view of a second embodiment of the retractable flow maintaining stent 14 of the present invention is shown positioned within its own constraining sheath 16a that is within the catheter sheath 16 as shown in FIG. 1 awaiting deployment. In this second embodiment, the micro-stent 14 may be contained within a secondary covering sheath 16a, which is contained in the outer catheter sheath 16. The micro-stent 14 may be inserted as attached to a guide wire 12 to remove an obstruction in a very small vessel, such as the intracranial vessels. Further, the micro-stent 14 may be deployed by pushing through the obstruction and expanding in order to pin the material 42 against the walls of the vessel 40 while still allowing blood to flow through the interlaced metallic mesh material of the stent 14, instead of pulling the stent 14 away from the affected site. The stent 14 expands in the same way whether two sheaths 16, 16a are employed, or just a single sheath 16 is employed. The cylindrical section 26 remains in the collapsed state until it has substantially exited the end of the sheath 16a such that it will deploy or expand in stages as the constraints of the sheath 16a are removed. Once the flexible joint 25 has exited the sheath 16a the distal section 26 has almost completely formed its cylindrical form at its full radial expansion. With the proximal section 24 exiting the sheath 16a and forming its full conical form the stent 14 is considered to be fully expanded. The sheath 16a can be withdrawn or the stent 14 can be collapsed and pushed forward as desired. Withdrawal of the stent 14 from the treatment site is also the same with the conical section 24 being drawn into the sheath 16a first causing the collapse of the conical section 24 followed by the flexible joint 25 which causes the staged collapse of the cylindrical section 26 as the stent 14 is drawn farther into the sheath 16a. The micro-stent 14 can then be repositioned for further treatment procedures or withdrawn entirely from the affected area.

In the event that the stent 14 becomes engaged with a vessel wall or for some other reason it cannot be retrieved or retracted into the sheath 16, 16a, then the flexible joint 25 becomes extremely important to the continuing viability of the vessel and ultimately the patient being treated. The flexible joint is manufactured of a material that has a lesser overall tensile strength than the materials utilized for the cylindrical section 26 and the conical section 24 of the stent 14. If the stent 14 becomes entangled in the vessels, engaged with a vessel wall such that a tear is imminent, or is otherwise not capable of being removed, then the better medical practice would be to let the stent 14 remain in place in the vessel. However, the conical section 24 by its flow restrictive conical structure, could be an impediment to the free flow of bodily fluids that the stent 14 was inserted to repair. Such is not the case with the cylindrical section 26 which, when is expanded against the vessel wall, has little or no flow impediments. In these circumstances the two sections 24, 26 of the stent 14 can be separated at the flexible joint 25 through the use of a twisting motion by rotating the conical section 24 either clockwise or counterclockwise to break the two sections 24, 26 apart at the flexible joint 25. The flexible joint will break before either the conical section 24 or the cylindrical section 26 due to the material of the flexible joint 25 having a reduced tensile strength than the materials of the conical and cylindrical sections 24, 26. By breaking the stent 14 into parts, the conical section 24 can be withdrawn and the cylindrical section 26 left in place without any considerable disruption to fluid flow within the vessel and little or no damage to the vessel or artery.

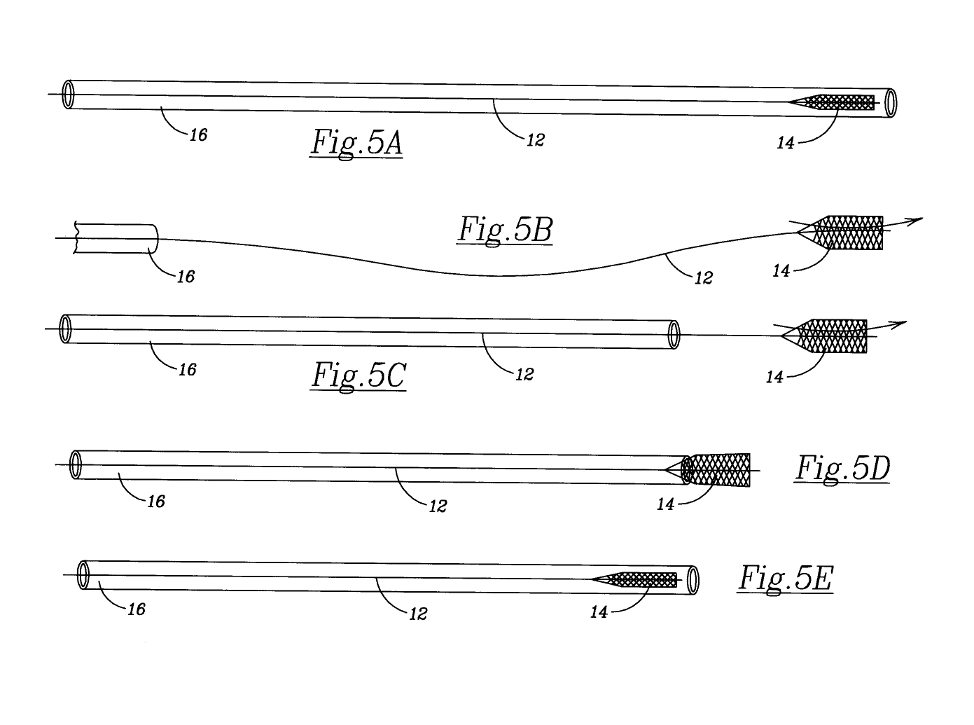

The various deployment options of the stent 14 are shown in FIGS. 5A, 5B, 5C, 5D and 5E. FIG. 5A shows a plan view of an elongated catheter sheath 16 containing a constrained stent 14 attached to a length of guide wire 12 such that the stent 14 is poised to be used once the sheath is positioned adjacent to the affected site for treatment. The guide-wire 12 may be advanced inside the elongated catheter sheath 16 pushing the micro-stent 14 outward from the distal end of the sheath 16. Shown in FIG. 5B is a plan view of the stent 14 with attached guide wire 12 of the present invention in a deployed state with the constraining sheath 16 withdrawn from along almost the entire length of the guide wire 12. The covering sheath 16 is pulled away to allow the expansion of the stent 14 against the vessel walls and to pin the biological material causing the blockage against the walls and allow blood flow through the mesh material of stent 14 as shown by the flow arrow.

FIG. 5C is a plan view showing the stent 14 in its deployed, expanded state proximate to the distal end of the constraining sheath 16 and attached to the guide wire 12. FIG. 5D is the next in the series of drawing figures showing the staged retraction of the stent 14 of the present invention. FIG. 5D is a plan view of the initiation of the recapture of the stent 14 by retracting the guide wire 12 causing the stent 14 to be drawn inward and re-enter the constraining sheath 16. A partial collapse of the conical section will begin causing a more extensive collapse as the stent 14 is drawn in farther along the sheath 16. FIG. 5E is the final plan view of the stent 14 of the present invention showing the guide wire 12 withdrawn into the sheath 16 and the stent 14 recaptured and collapsed within the constraining sheath 16 ready for redeployment or withdrawal.

Several uses for the stent 14 and its cooperating sheath 16 are readily obtainable in the field of vascular and micro-vascular surgery. Revascularization of the cerebro-vascular bed is achieved using the flow maintaining stent 14 of the present invention. The treatment protocol includes obtaining arterial access and utilizing a wire exchange, whereby a short sheath is positioned in the arterial vessel. A pigtail catheter is positioned with wire guidance and an arch angiography is performed to determine the position and mass of the occlusion. A 0.035 guide wire is placed into the pigtail catheter and it is removed. Selective angiography of the vasculature is performed in accepted fashion utilizing a commercially available shaped catheter which may be manufactured by Vitek, Simmons, Headhunter, Vertebral, etc. The culprit vessel is then selectively entering using a specific commercially available guide-wire, such as Glidewire, Magic Torque or Supercore, and the like, manufactured by a number of companies. This guide wire is used to place a non-selective sheath 16 into the culprit vessel such that the sheath extends distally from a point in the culprit vessel, e.g., one of the common carotids or vertebral arteries, to the proximal end of the sheath at the groin entry site where the hemostatic valve is located. Once the sheath is satisfactorily placed in the main cervical vessel, a 0.014 guide wire 12 carrying the stent 14 is advanced through the sheath 16 into the intracranial vessel to the point of obstruction. The guide wire 12 and stent 14 can be advanced alone or with the use of a micro-catheter 16a for support. Either the guide wire 12 and stent 14 are advanced across the obstruction or, if supported by a micro-catheter 16a, all are deployed forward across the obstruction. This is followed by crossing the obstruction over the guide wire 12 and stent 14 with the micro-catheter 16a for retracting the guide wire 12 and stent 14 for repositioning. The procedure is repeated until flow is restored.

Once flow is restored at the site of the obstruction by fluid flow through the stent 14 of the present invention, the stent 14 may be recaptured by withdrawing it into the micro-catheter 16a such that it collapses into it stored position within the sheath. The micro-catheter 16a can either be retracted into the larger sheath or removed. Alternatively, if the guide wire 12 and stent are advanced without a micro-catheter 16a, the sheath 16 containing the guide wire 12 and stent 14 is placed across the obstruction. The covering sheath 16 is retracted or removed exposing the stent 14 at the distal end of the guide wire 12. Proper placement of the stent 14 will span the obstruction, immediately pinning the obstructing material aside and allowing flow to be restored through the mesh material of the stent 14. If the stent 14 of the device contains a drug coating, the obstructing material being pinned behind the stent 14 against the vessel wall will be in direct contact with the drug containing stent struts. Upon restoring flow or reaching drug elution time, the cover/constraining sheath 16 is re-advanced to recapture the stent 14 and the re-constrained stent 14 can be removed. Angiography determines if the procedure is finished or if further work such as balloon dilation, permanent stent placement or additional use of the stent 14 of the present invention in a more distal segment in the vessel is necessary. A post-procedural angiography is performed and the long sheath is removed and either replaced at the groin site with a short sheath or the standard sheath removal procedure is followed.

Another area in which the guide wire 12 and stent 14 of the present invention may be used is in the revascularization of the pulmonary arterial bed. Revascularization of the pulmonary arterial bed is achieved using the flow maintaining stent 14 of the present invention. The treatment protocol includes the placing of a standard sheath in the femoral vein. A 0.035 guide-wire is advanced to the right atrium. A commercially available pre-shaped catheter such as Judkins Right 4, LIMA, etc. is advanced into the right atrium. The 0.035 wire is manipulated through the right ventricle to the pulmonary artery, or alternatively, a Swan Ganz catheter with a 0.021 wire is manipulated to the pulmonary artery and placed over the 0.021 wire, then the Swan-Ganz catheter is withdrawn and a catheter with a larger internal diameter is introduced over the 0.021 wire, the 0.021 wire is removed and a 0.035 wire is introduced and the catheter is removed leaving only the 0.035 wire. A pigtail catheter is placed over the 0.035 wire and then the wire is removed. A pulmonary angiography is performed and the 0.035 wire is replaced and the pigtail catheter removed. Advance an end hole catheter such as a Multipurpose, RCA, Vert and the like over the 0.035 wire. Advance the end hole catheter into or beyond the thrombus/obstruction and remove the 0.035 wire. Insert and advance the guide wire 12 and stent 14 through the catheter/sheath 16 to the furthest distal portion of the catheter/sheath 16. Pull back or retract the catheter/sheath 16 (end-hole catheter) allowing the stent 14 to be deployed and expand. Alternatively, advance the covered stent 14 at the distal end of the guide wire 12 just beyond the end of the catheter/sheath 16 and remove the end-hole catheter 16 leaving just the stent-wire system 12, 14 within the micro-catheter 16a. Retract the sheath 16a by pulling it back into the main catheter so that the stent 14 deploys and is permitted to expand. Re-advance the end-hole catheter to the main pulmonary artery and perform a Puff angiogram to evaluate flow. Re-constrain the stent 14 by pushing the micro-catheter 16a over the expanded form or by retracting and collapsing the stent 14 as described above upon completion of dwell time or drug elution. Re-advance the end-hole catheter, remove the stent 14 and replace the 0.035 guide-wire. Retract the end-hole catheter to main pulmonary artery and perform a Puff or standard angiography depending on the catheter. Based upon the angiogram the surgeon may elect to perform one or more of the following: re-advance and re-treat the same area/vessel, re-direct the wire into another segment and treat, re-direct into the other pulmonary artery and treat, or removal. Once the treatment protocol is completed, remove the end-hole catheter and the wire. Replace the pigtail catheter and remove the wire and perform a final pulmonary angiography.

Another vascular treatment protocol may be as described immediately following. Revascularization of the coronary bed is achieved using the flow maintaining stent 14 of the present invention. The treatment protocol includes a standard sheath placement and the advancing of a guide-wire and shaped guide catheter, in standard fashion, to allow standard selective coronary angiography to identify the narrowing or obstruction, and quantify the degree of narrowing or obstruction, as well as the dimensions of the parent vessel. Advance the constrained stent 14 through the sheath 16 and across the vessel narrowing or obstruction. Deploy the stent 14 in its expanded state such that it placement spans from beyond the obstruction distally to before the obstruction proximally. This positioning will pin the obstructing material between the stent 14 and the vessel wall, while flow is maintained within the stent 14 by entering through the partially open proximal interstices of the mesh material and exiting through the lumen beyond the stent 14. On the outer margin of the expanded stent 14, the material pinned will either remain pinned or dissolve by the natural anticoagulant properties of the flow. If the stent 14 is drug coated, the drug on the outside surface of the stent 14 will be in direct contact with the obstructing material and assist in the anti-coagulation. When drug elution time is complete, re-constrain the stent 14 by retracting it into the sheath 16 causing the collapse of the stent 14 and remove from the treatment area. Alternatively, if the re-constraining sleeve or sheath 16 is completely removable then advance it and remove the stent 14 and its associated components completely. Position a standard 0.014 coronary guide-wire in the sheath and remove the constraining sheath 16a. Perform a coronary angiogram to assess the therapeutic success of the flow maintaining stent 14. If a heavy thrombus or obstruction remains, continue with standard therapy.

The suggested overall length dimension of the micro-stent 14 is in the range of approximately 15 mm to 30 mm, but would be preferred to be 20 mm in length. The conical section 24 is suggested to extend approximately 25% to 33% of the overall length of the stent 14. These suggested dimensions will afford a sufficient area for compressive treatment of the obstruction or occlusion and permit the unfettered deployment and retraction of the micro-stent 14 within the vessel. The guide wire 12 is also preferred to be of the type having the property or characteristic of retaining a shaped, e.g., a bending, so as to traverse a difficult course around a bend or curve in the artery or vessel. The forward extension of the guide wire 12 beyond the distal end of the collapsed stent 14 is preferred to be in the range of approximately 10 mm, but could be longer or shorter.

In the event that the stent wire 12 breaks at or near its joint 22 with the stent 14 while deployed, a snare wire can be introduced into the sheath 16 to recapture the stent 14 and broken wire 12 by grabbing the conical section 24 forward of the joint with the wire 12 and pushing the sheath 16 forward over the conical section 24 forcing its collapse and the subsequent forced collapse of the cylindrical section 26. Once the broken stent wire 12 with attached micro stent 14 is within the sheath, they can be safely removed from the treatment area without injury to the vessel or artery. This break and recapture is different than the intentional separation of the proximal and distal sections of the micro-stent 14 at the flexible junction 25 between the conical and cylindrical sections 24, 26 described above. In that instance, the cylindrical section 26 is intended to remain as it is not retrievable without damage to the vessel or artery, while the conical section 24 is retrieved and removed.

The present invention may be embodied in other specific forms without departing from the spirit or essential attributes thereof and, accordingly, the described embodiments are to be considered in all respects as being illustrative and not restrictive, with the scope of the invention being indicated by the appended claims, rather than the foregoing detailed description, as indicating the scope of the invention as well as all modifications which may fall within a range of equivalency which are also intended to be embraced therein.

1. A retractable self-expanding stent apparatus for re-establishing fluid flow in a body vessel while maintaining fluid flow within the vessel during use comprising:
a self-expanding stent having a proximal and distal end and being partitioned into a full expansion portion and a partial expansion portion, said full expansion portion configured as a cylindrical section and located at the distal end of the stent, and said partial expansion portion configured as a conical section and located at the proximal end of the stent;
the cylindrical and the conical sections are joined at their juxtaposed ends by a common flexible reinforcing joint, which joint permits the independent staged expansion of the cylindrical portion to its full diametric expansion prior to and continuing as the conical section reaches full expansion and which joint permits the collapse of the conical section prior to and continuing through the staged collapse of the cylindrical section, said common joint having the same maximum diametric dimension for both the cylindrical section and the conical section of the stent;
a guide wire attached to the proximal end of the stent for controlling the positioning of the stent;
at least one covering sheath for retaining and transporting the self-expanding stent and guide wire.

2. The apparatus of claim 1, wherein said stent has a predetermined geometry of cross-wire mesh-like configuration having sufficient interstitial spacing to permit the re-establishment of bodily fluid flow depending upon the area of use within the human body.

3. The apparatus of claim 1, wherein said stent is capable of self-expansion upon deployment and self-collapse upon retraction as said stent is respectively pushed out of and pulled into the covering sheath.

4. The apparatus of claim 1, wherein said stent is capable of self-collapse upon retraction as said covering sheath is pushed over the stent and self-expansion upon deployment as the stent is pushed out of said covering sheath.

5. The apparatus of claim 1 further comprising a tip located at the distal end of the stent wire capable of penetrating a blockage or obstruction in a vessel.

6. The apparatus of claim 1, wherein said stent is capable of expanding entirely across a vessel at deployment causing the material of the blockage to be pushed against the vessel walls and to immediately permit the re-establishment of bodily fluid flow again beyond the point of obstruction.

7. The apparatus of claim 1, wherein the common flexible reinforcing joint has a lesser tensile strength than the conical or cylindrical sections of the stent such that a rotating motion will cause the conical and cylindrical sections to separate at the flexible joint.

8. The apparatus of claim 1, wherein said stent is capable of being coated with one or more pharmaceutical agents to locally treat the obstruction in a vessel.

9. A method for the deployment and recapture of a retractable self-expanding stent apparatus for re-establishing fluid flow in a body vessel while maintaining fluid flow within the vessel during use comprising the steps of:
positioning a self-expanding stent having a proximal and distal end and being partitioned into a full expansion portion and a partial expansion portion, said full expansion portion configured as a cylindrical section and located at the distal end of the stent, and said partial expansion portion configured as a conical section and located at the proximal end of the stent, said cylindrical and conical sections being joined at their juxtaposed ends by a common flexible reinforcing joint, which joint permits the independent staged expansion of the cylindrical portion to its full diametric expansion prior to and continuing as the conical section reaches full expansion and which joint permits the collapse of the conical section prior to and continuing through the staged collapse of the cylindrical section, such that the common joint has the same maximum diametric dimension for both the cylindrical section and the conical section of the stent, and a guide wire attached to said proximal end of the conical section of said stent inside a catheter or sheath;
positioning the sheath with said stent and guide wire attached at the point of obstruction in a vessel containing bodily fluids of a human;
advancing the guide wire outward from the distal end of the sheath, exposing the cylindrical section of the stent beyond the common joint so that said stent will begin its self-expansion as the conical section is further exposed and is permitted to expand beyond the diametric confining dimension of said sheath; and,
withdrawing the guide wire inward away from the distal end of the sheath, contacting the conical section of the stent against the distal opening of the sheath and forcing the reduction in the diametric measurement of the common joint by the sliding contact of the conical section of the stent against the inner circumference of the sheath resulting in the collapse of the stent to a size capable of being completely drawn into the sheath.

10. The method of claim 9 further comprising the step of placing the stent and connected guide wire positioned inside the sheath within another catheter or sheath.

11. The method of claim 9 further comprising the step coating the stent with one or more pharmaceutical agents to locally treat the obstruction in a vessel.

12. The method of claim 9 further comprising the step of locating an extended tip at the distal end of the stent wire capable of penetrating a blockage or obstruction in a vessel.

13. The method of claim 9 further comprising the step of separating the conical and cylindrical sections of the stent by a rotating motion such that the separation occurs at the common flexible joint.

14. A method for the deployment and recapture of a retractable self-expanding stent apparatus for re-establishing fluid flow in a body vessel while maintaining fluid flow within the vessel during use comprising the steps of:
positioning a self-expanding stent having a proximal and distal end and being partitioned into a full expansion portion and a partial expansion portion, said full expansion portion configured as a cylindrical section and located at the distal end of the stent, and said partial expansion portion configured as a conical section and located at the proximal end of the stent, said cylindrical and conical sections being joined at their juxtaposed ends by a common reinforcing joint, which joint permits the independent staged expansion of the cylindrical portion to its full diametric expansion prior to and continuing as the conical section reaches full expansion and which joint permits the collapse of the conical section prior to and continuing through the staged collapse of the cylindrical section, such that the common joint has the same maximum diametric dimension for both the cylindrical section and the conical section of the stent, and a guide wire attached to said proximal end of the conical section of said stent inside a catheter or sheath;
positioning the sheath with said stent and guide wire attached at the point of obstruction in a vessel containing bodily fluids of a human;
pulling the sheath back and away from the stent along the guide wire, exposing the cylindrical section of the stent beyond the common joint so that said stent will begin its self-expansion as the conical section is further exposed and is permitted to expand beyond the diametric confining dimension of said sheath; and,
pushing the sheath along the guide wire, contacting the conical section of the stent against the distal opening of the sheath and forcing the reduction in the diametric measurement of the common joint by the sliding contact of the conical section of the stent against the inner circumference of the sheath resulting in the collapse of the stent to a size capable of being completely drawn into the sheath.

15. The method of claim 14 further comprising the step of placing the stent and connected guide wire positioned inside the sheath within another catheter or sheath.

16. The method of claim 14 further comprising the step coating the stent with one or more pharmaceutical agents to locally treat the obstruction in a vessel.

17. The method of claim 14 further comprising the step of locating an extended tip at the distal end of the stent wire capable of penetrating a blockage or obstruction in a vessel.

18. The method of claim 14 further comprising the step of separating the conical and cylindrical sections of the stent by a rotating motion such that the separation occurs at the common flexible joint.
